(19) United States
(12) Patent Application Publication
(10) Pub. No.: US 2024/0350428 A1
(43) Pub. Date: Oct. 24, 2024

(54) USE OF CANNABINOIDS IN THE TREATMENT OF EPILEPSY

(71) Applicant: Jazz Pharmaceuticals Reseach UK Limited, Sittingbourne (GB)

(72) Inventors: Geoffrey GUY, Cambridge (GB); Stephen Wright, Altrincham (GB); Orrin Devinsky, New York, NY (US)

(21) Appl. No.: 18/762,588

(22) Filed: Jul. 2, 2024

Related U.S. Application Data
(63) Continuation of application No. 17/744,224, filed on May 13, 2022, now Pat. No. 12,064,399.
Application No. 17/744,224 is a continuation of application No. 16/893,018, filed on Jun. 4, 2020, now Pat. No. 11,357,741.
Application No. 16/893,018 is a continuation of application No. 15/183,947, filed on Jun. 16, 2016, now Pat. No. 10,709,671.

(30) Foreign Application Priority Data
Jun. 17, 2015 (GB) 1510664.4

(51) Int. Cl. A61K 31/05; A61K 31/352; A61K 31/5513; A61K 36/185; A61K 45/06; A61K 47/10; A61K 47/26; A61K 47/44; A61P 25/08

(52) U.S. Cl. CPC A61K 31/05; A61K 31/352; A61K 31/5513; A61K 36/185; A61K 45/06; A61K 47/10; A61K 47/26; A61K 47/44; A61P 25/08

(57) ABSTRACT

The present invention relates to the use of cannabidiol (CBD) in the treatment of focal seizures. In one embodiment the patients suffering from focal seizures are children and young adults. CBD appears particularly effective in reducing focal seizures in patients suffering with etiologies that include: Lennox-Gastaut Syndrome; Tuberous Sclerosis Complex; Dravet Syndrome; CDKL5; Neuronal ceroid lipofuscinoses (NCL); febrile infection related epilepsy syndrome (FIRES); Aicardi syndrome and brain abnormalities in comparison to other seizure types. Significantly CBD additionally is very effective in the reduction of a sub-type of focal seizures, focal seizures with impairment.

### FIELD OF THE INVENTION

The present invention relates to the use of cannabidiol (CBD) in the treatment of focal seizures. In one embodiment the patients suffering from focal seizures are children and young adults. CBD appears particularly effective in reducing focal seizures in patients suffering with etiologies that include: Lennox-Gastaut Syndrome; Tuberous Sclerosis Complex; Dravet Syndrome; CDKL5; Neuronal ceroid lipofuscinoses (NCL); febrile infection related epilepsy syndrome (FIRES); Aicardi syndrome and brain abnormalities in comparison to other seizure types.

Significantly CBD additionally is very effective in the reduction of a sub-type of focal seizures, focal seizures with impairment. The etiologies of patients which suffer from focal seizures with impairment include: Lennox-Gastaut Syndrome; Tuberous Sclerosis Complex; Dravet Syndrome; CDKL5; febrile infection related epilepsy syndrome (FIRES); Aicardi syndrome and brain abnormalities.

In these patients treatment with CBD reduced the occurrence of absence seizures or myoclonic absence seizures by greater than 50% in a large proportion of patients, 64% and 75% respectively. This was surprising given that the proportion of patients benefitting from a greater than 50% reduction in total seizures was significantly less, (46%), in all subjects treated.

Preferably the CBD used is in the form of a highly purified extract of Cannabis such that the CBD is present at greater than 98% of the total extract (w/w) and the other components of the extract are characterised. In particular the cannabinoid tetrahydrocannabinol (THC) has been substantially removed, to a level of not more than 0.15% (w/w) and the propyl analogue of CBD, cannabidivarin, (CBDV) is present in amounts of up to 1%. Alternatively, the CBD may be a synthetically produced CBD.

In use the CBD may be used concomitantly with one or more other anti-epileptic drugs (AED). Alternatively the CBD may be formulated for administration separately, sequentially or simultaneously with one or more AED or the combination may be provided in a single dosage form. Where the CBD is formulated for administration separately, sequentially or simultaneously it may be provided as a kit or together with instructions to administer the one or more components in the manner indicated. It may also be used as the sole medication, i.e. as a monotherapy.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom using the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment-resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

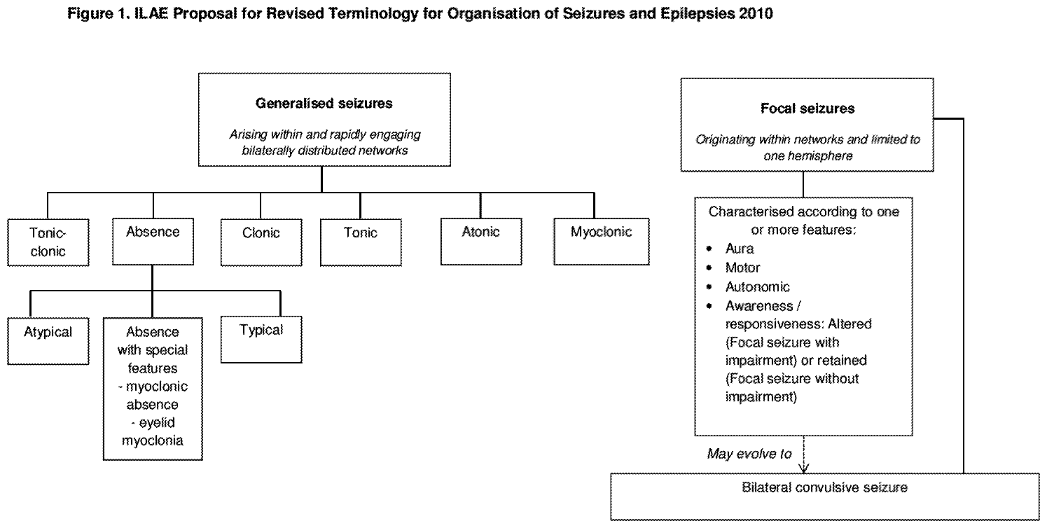

The main symptom of epilepsy is repeated seizures. In order to determine the type of epilepsy or the epileptic syndrome that a patient is suffering from, an investigation into the type of seizures that the patient is experiencing is undertaken. Clinical observations and electroencephalography (EEG) tests are conducted and the type(s) of seizures are classified according to the ILAE classification described below and in FIG. 1.

The International classification of seizure types proposed by the ILAE was adopted in 1981 and a revised proposal was published by the ILAE in 2010 and has not yet superseded the 1981 classification. FIG. 1 is adapted from the 2010 proposal for revised terminology and includes the proposed changes to replace the terminology of partial with focal. In addition the term “simple partial seizure” has been replaced by the term “focal seizure where awareness/responsiveness is not impaired” and the term “complex partial seizure” has been replaced by the term “focal seizure where awareness/consciousness is impaired”.

From FIG. 1 it can be seen that Generalised seizures, where the seizure arises within and rapidly engages bilaterally distributed networks, can be split into six subtypes: Tonic-Clonic (grand mal) seizures; Absence (petit mal) Seizures; Clonic Seizures; Tonic Seizures; Atonic Seizures and Myoclonic Seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a Bilateral convulsive seizure, which is the proposed terminology to replace Secondary Generalized Seizures (generalized seizures that have evolved from focal seizures and are no longer remain localized).

Focal seizures where the subjects awareness/responsiveness is altered are referred to as focal seizures with impairment and focal seizures where the awareness or responsiveness of the subject is not impaired are referred to as focal seizures without impairment.

Focal seizures may occur in epilepsy syndromes including: Lennox-Gastaut Syndrome; Tuberous Sclerosis Complex; Dravet Syndrome; CDKL5; Neuronal ceroid lipofuscinoses (NCL); febrile infection related epilepsy syndrome (FIRES); Aicardi syndrome and brain abnormalities.

Epileptic syndromes often present with many different types of seizure and identifying the types of seizure that a patient is suffering from is important as many of the standard AED's are targeted to treat or are only effective against a given seizure type/sub-type.

One such childhood epilepsy is Dravet syndrome. Onset of Dravet syndrome almost always occurs during the first year of life with clonic and tonic-clonic seizures in previously healthy and developmentally normal infants (Dravet, 2011). Symptoms peak at about five months of age. Other seizures develop between one and four years of age such as prolonged focal dyscognitive seizures and brief absence seizures.

In diagnosing Dravet syndrome both focal and generalised seizures are considered to be mandatory, Dravet patients may also experience atypical absence seizures, myoclonic absence seizures, atonic seizures and non-convulsive status epilepticus.

Seizures progress to be frequent and treatment-resistant, meaning that the seizures do not respond well to treatment. They also tend to be prolonged, lasting more than 5 minutes. Prolonged seizures may lead to status epilepticus, which is a seizure that lasts more than 30 minutes, or seizures that occur in clusters, one after another.

Prognosis is poor and approximately 14% of children die during a seizure, because of infection, or suddenly due to uncertain causes, often because of the relentless neurological decline. Patients develop intellectual disability and life-long ongoing seizures. Intellectual impairment varies from severe in 50% patients, to moderate and mild intellectual disability each accounting for 25% of cases.

There are currently no FDA approved treatments specifically indicated for Dravet syndrome. The standard of care usually involves a combination of the following anticonvulsants: clobazam, clonazepam, levetiracetam, topiramate and valproic acid.

Stiripentol is approved in Europe for the treatment of Dravet syndrome in conjunction with clobazam and valproic acid. In the US, stiripentol was granted an Orphan Designation for the treatment of Dravet syndrome in 2008; however, the drug is not FDA approved.

Potent sodium channel blockers used to treat epilepsy actually increase seizure frequency in patients with Dravet Syndrome. The most common are phenytoin, carbamazepine, lamotrigine and rufinamide.

Management may also include a ketogenic diet, and physical and vagus nerve stimulation. In addition to anti-convulsive drugs, many patients with Dravet syndrome are treated with anti-psychotic drugs, stimulants, and drugs to treat insomnia.

The first line treatment for focal seizures are AED such as carbamezapine or lamotrigine. Levetiracetam, oxycarbamezapine or sodium valproate are also considered to be of use. A combination of these medicaments may be required in order to treat focal seizures.

Common AED defined by their mechanisms of action are described in the following tables:

TABLE 1
Examples of narrow spectrum AED

| Narrow- | | |
|---|---|---|
| spectrum | | |
| AED | Mechanism | Indication |
| Phenytoin | Sodium channel | Complex partial |
| | | Tonic-clonic |
| Phenobarbital | GABA/Calcium channel | Partial seizures |
| | | Tonic-clonic |
| Carbamazepine | Sodium channel | Partial seizures |
| | | Tonic-clonic |
| | | Mixed seizures |
| Oxcarbazepine | Sodium channel | Partial seizures |
| | | Tonic-clonic |
| | | Mixed seizures |
| Gabapentin | Calcium channel | Partial seizures |
| | | Mixed seizures |
| Pregabalin | Calcium channel | Adjunct therapy for partial |
| | | seizures with or without |
| | | secondary generalisation |
| Lacosamide | Sodium channel | Adjunct therapy for partial |
| | | seizures |
| Vigabatrin | GABA | Secondarily generalized tonic- |
| | | clonic seizures |
| | | Partial seizures |
| | | Infantile spasms due to West |
| | | syndrome |

TABLE 2
Examples of broad spectrum AED

| Broad- | | |
|---|---|---|
| spectrum | | |
| AED | Mechanism | Indication |
| Valproic acid | GABA/Sodium channel | First-line treatment for tonic- |
| | | clonic seizures, absence |
| | | seizures and myoclonic |
| | | seizures |
| | | Second-line treatment for |
| | | partial seizures and infantile |
| | | spasms. |
| | | Intravenous use in status |
| | | epilepticus |
| Lamotrigine | Sodium channel | Partial seizures |
| | | Tonic-clonic |
| | | Seizures associated with |
| | | Lennox-Gastaut syndrome |
| Ethosuximide | Calcium channel | Absence seizures |
| Topiramate | GABA/Sodium channel | Seizures associated with |
| | | Lennox-Gastaut syndrome |
| Zonisamide | GABA/Calcium/Sodium | Adjunctive therapy in adults |
| | channel | with partial-onset seizures |
| | | Infantile spasm |
| | | Mixed seizure |
| | | Lennox-Gastaut syndrome |
| | | Myoclonic |
| | | Generalised tonic-clonic |
| | | seizure |
| Levetiracetam | Calcium channel | Partial seizures |
| | | Adjunctive therapy for partial, |
| | | myoclonic and tonic-clonic |
| | | seizures |
| Clonazepam | GABA | Typical and atypical absences |
| | | Infantile myoclonic |
| | | Myoclonic seizures |
| | | Akinetic seizures |
| Rufinamide | Sodium channel | Adjunctive treatment of |
| | | partial seizures associated |
| | | with Lennox-Gastaut |
| | | syndrome |

TABLE 3
Examples of AED used specifically in childhood epilepsy

| AED | Mechanism | Indication |
|---|---|---|
| Clobazam | GABA | Adjunctive therapy in complex |
| | | partial seizures |
| | | Status epilepticus |
| | | Myoclonic |
| | | Myoclonic-absent |
| | | Simple partial |
| | | Complex partial |
| | | Absence seizures |
| | | Lennox-Gastaut syndrome |
| Stiripentol | GABA | Severe myoclonic epilepsy in |
| | | infancy (Dravet syndrome) |

From these tables it can be seen that there are many AED are approved for use in focal (partial) seizures which work by a different mechanisms. Indeed the only AED that has been approved for use in the treatment of complex partial seizures (focal seizures with impairment) is the AED phenytoin.

Over the past forty years there have been a number of animal studies on the use of the non-psychoactive cannabinoid cannabidiol (CBD) to treat seizures. For example, Consroe et al., (1982) determined that CBD was able to prevent seizures in mice after administration of pro-convulsant drugs or an electric current.

Studies in epileptic adults have also occurred in the past forty years with CBD. Cunha et al. reported that administration of CBD to eight adult patients with generalized epilepsy resulted in a marked reduction of seizures in 4 of the patients (Cunha et al., 1980).

A study in 1978 provided 200 mg/day of pure CBD to four adult patients, two of the four patients became seizure free, whereas in the remainder seizure frequency was unchanged (Mechoulam and Carlini, 1978).

In contrast to the studies described above, an open label study reported that 200 mg/day of pure CBD was ineffective in controlling seizures in twelve institutionalized adult patients (Ames and Cridland, 1986).

Based on the fact that chronologically the last study to look at the effectiveness of CBD in patients with epilepsy proved that CBD was unable to control seizures, there would be no expectation that CBD might be useful as an anti-convulsant agent.

In the past forty years of research there have been over thirty drugs approved for the treatment of epilepsy none of which are cannabinoids. Indeed, there appears to have been a prejudice against cannabinoids, possibly due to the scheduled nature of these compounds and/or the fact that THC, which is a known psychoactive, has been ascribed as a pro-convulsant (Consroe et al., 1977).

A paper published recently suggested that cannabidiol-enriched Cannabis may be efficacious in the treatment of epilepsy. Porter and Jacobson (2013) report on a parent survey conducted via a Facebook group which explored the use of Cannabis which was enriched with CBD in children with treatment-resistant epilepsy. It was found that sixteen of the 19 parents surveyed reported an improvement in their child's epilepsy. The children surveyed for this paper were all taking Cannabis that was purported to contain CBD in a high concentration although the amount of CBD present and the other constituents including THC were not known for many of the cases. Indeed, whilst CBD levels ranged from 0.5 to 28.6 mg/kg/day (in those extracts tested), THC levels as high as 0.8 mg/kg/day were reported.

Providing children with TRE with a Cannabis extract that comprises THC, which has been described as a pro-convulsant (Consroe et al., 1977), at a potentially psychoactive dose of 0.8 mg/kg/day, is a concern and as such there is a need to determine whether CBD is in fact efficacious.

In November 2013 the company GW Pharmaceuticals made a press release to state that they were intending to treat Dravet Syndrome with CBD as it had received orphan drug designation. A further press release was made in June 2014 which stated promising signals of efficacy in children with treatment-resistant epilepsy, including patients with Dravet syndrome.

The international patent application WO 2015/193667 describes the use of CBD in treatment resistant epilepsy. Patients included nine with Dravet syndrome out of 27 others.

The international patent application WO 2015/193668 describes the use of CBD in the treatment of absence seizures. Patients included those with Dravet syndrome in addition to ten other syndromes.

Maa and Figi (2014) discuss the case for medical marijuana in epilepsy and discuss the positive treatment of a girl Charlotte with Dravet syndrome who experienced frequent bouts of febrile and afebrile status epilepticus as well as tonic, tonic-clonic and myoclonic seizures (generalised seizures). She was given an extract from a Cannabis plant dubbed “Charlotte's Web” which according to the suppliers, CW Botanicals, disclose that their extracts are rich in terpenes and contain from 10 to 200 times the amount found in other proprietary plants. In other words the suggestion is that the efficacy is based on a combination of CBD and the terpenes present in their extracts.

Press et al. (3 Apr. 2015), provides an in depth review of the parental reporting of pediatric patients with refractory epilepsy that were given oral Cannabis extracts (OCE). Despite it's in depth nature it concludes no studies demonstrate clear efficacy.

Significantly the document recognizes the effectiveness of an anti-seizure medication may be dependent upon: the drug itself, including CBD, (see Table 3); the epilepsy syndrome type (Table 2); and the seizure type (Table 2).

Very significantly the document in the discussion recognises caution needs to be taken when reviewing, particularly open label study data, since placebo rates may be high. Indeed it specifically comments that “four recently FDA approved anti-convulsant medications had placebo rates of 31.6%, 26.4%, 20% and 21% respectively” (page 51, left hand column).

Furthermore the analysis observed a surprising finding namely that “new residents of Colorado (those moving to obtain treatment) were more than three times as likely to report a greater than 50% seizure reduction than families with established care in Colorado” suggestive that studies such as that published in Porter and Jacobson (2013) may be highly flawed.

The skilled person would infer therefore from Press et al. would be that the drug type CBD plus the presence of “other OCE” (such as, other cannabinoids most likely THC and non-cannabinoids such as e.g. terpenes) appears a more interesting combination than CBD alone—responder rate 63% versus 35%.

That the epilepsy syndrome Lennox-Gastaut appears the most promising target with 89% responder rate versus Dravet (23% responder rate) or Doose (0% responder rate).

That of the seizure types studied ranged from 44% responder rate for atonic seizures to 17% responder rates in tonic seizures, amongst the seven seizures types reviewed.

The assessment looked at three distinct groups, namely: the OCE type, Table 3 (four OCE types); the epilepsy syndrome, see for example, page 50 right hand column line 3 (three syndrome types); and the seizure type, see page 51, Table 2 (seven seizure types).

In all this provides the reader with information on 84 different alternative combinations.

The problem facing the skilled practitioner looking at Cannabis medicines in the field of epilepsy where many patients are refractory to existing medications is to select the appropriate cannabinoid and its form targeted to a given seizure type in a given patient group.

Perhaps therefore it is not surprising that in the Cochrane report (Gloss and Vickrey) published March 2014 undertook a full review on the efficacy of cannabinoids in the treatment of epilepsy concluded “no reliable conclusions can be drawn at present regarding the efficacy of cannabinoids as a treatment for epilepsy.”

Surprisingly the applicant has shown that CBD is particularly effective in the treatment of focal seizures in Dravet syndrome patients, particularly children and more particularly those which are resistant to existing treatments.

### BRIEF SUMMARY OF THE DISCLOSURE

In accordance with a first aspect of the present invention there is provided Cannabidiol (CBD) for use in the treatment of focal seizures in Dravet Syndrome.

In one embodiment the focal seizures are focal seizures with impairment.

Preferably the Dravet Syndrome is treatment-resistant.

In a further embodiment the CBD is for use in combination with one or more concomitant anti-epileptic drugs (AED).

In a further embodiment the CBD is present as a highly purified extract of Cannabis which comprises at least 98% (w/w) CBD. Preferably the extract comprises less than 0.15% THC. More preferably the extract further comprises up to 1% CBDV.

In an alternative embodiment the CBD is present as a synthetic compound.

In a further embodiment of the invention the one or more AED is selected from the group consisting of: carbamezapine, clobazam, clonazepam, clonidine, clorazepate, desmethylclobazam, diazepam, ethosuximide, felbamate, ketogenic diet, lacosamide, lamotrigine, levetiracetam, lorazepam, midazolam, N-desmethylclobazam, nordiazepam, oxycarbamezapine, perampanel, phenobarbital, phenytoin, pregabalin, rufinamide, stiripentol, topiramate, trazodone, vagus nerve stimulation, valproic acid, vigabatrin, and zonisamide.

Preferably the number of different anti-epileptic drugs that are used in combination with the CBD is reduced. Alternatively the dose of anti-epileptic drugs that are used in combination with the CBD is reduced.

There are many side effects associated with the commonly used AED which include dizziness, blurred vision, nausea, respiratory system depression, tiredness, headaches, and other motor side effects on the central nervous system. These side effects are particularly common as higher doses or combinations of numerous AED are used. As such there is a need for an alternative medication that is able to reduce the numbers of seizures whilst at the same time exhibiting a safe side effect profile.

Preferably the dose of CBD is greater than 5 mg/kg/day. Thus for a 15 kg patient a dose of greater than 75 mg of CBD per day would be provided. Doses greater than 5 mg/kg/day such as greater than 10/mg/kg/day, greater than 15 mg/kg/day, greater than 20 mg/kg/day and greater than 25 mg/kg/day are also envisaged to be effective.

In accordance with a second aspect of the present invention there is provided a method of treating focal seizures in Dravet Syndrome comprising administering cannabidiol (CBD) to a subject.

Preferably the subject is a human.

In accordance with a third aspect of the present invention there is provided a composition for use in the treatment of epilepsy characterised by focal seizures in Dravet syndrome comprising cannabidiol (CBD), a solvent, a co-solvent, a sweetener, and a flavouring.

Preferably the solvent is sesame oil, the co-solvent is ethanol, the sweetener is sucralose, the flavouring is strawberry flavour and the CBD is present at a concentration of between 25/mg/ml and 100 mg/ml, namely 50 mg/ml and 75 mg/ml.

More preferably the composition comprises cannabidiol (CBD) at a concentration of between 25 to 100 mg/ml, ethanol at a concentration of 79 mg/ml, sucralose at a concentration of 0.5 mg/ml, strawberry flavouring at a concentration of 0.2 mg/ml and sesame oil q.s. to 1.0 ml.

It is envisaged that the composition be administered as an oral liquid solution. Other modes of administration including solids, semi-solids, gels, sprays, aerosols, inhalers, vaporisers, enemas and suppositories are alternative administration forms. Such medicaments could be administered via the oral, buccal, sublingual, respiratory, nasal and distal rectum route.

### Definitions

Definitions of some of the terms used to describe the invention are detailed below.

The cannabinoids described in the present application are listed below along with their standard abbreviations.

TABLE 4
Cannabinoids and their abbreviations

| | | |
|---|---|---|
| CBD | Cannabidiol | 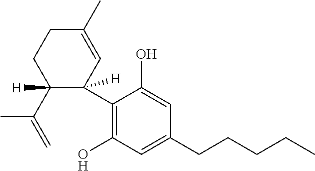 [SMILES: C=C(C)[C@@H]1CCC(C)=C[C@H]1c1c(O)cc(CCCCC)cc1O] |
| CBDA | Cannabidiolic acid | 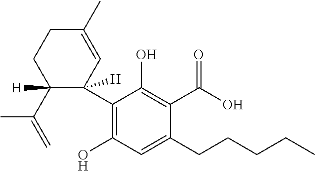 [SMILES: C=C(C)[C@@H]1CCC(C)=C[C@H]1c1c(O)cc(CCCCC)c(C(=O)O)c1O] |
| CBDV | Cannabidivarin | 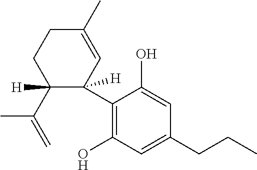 [SMILES: C=C(C)[C@@H]1CCC(C)=C[C@H]1c1c(O)cc(CCC)cc1O] |
| CBDVA | Cannabidivarinic acid | 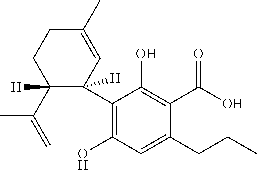 [SMILES: C=C(C)[C@@H]1CCC(C)=C[C@H]1c1c(O)cc(CCC)c(C(=O)O)c1O] |
| THC | Tetrahydrocannabinol | 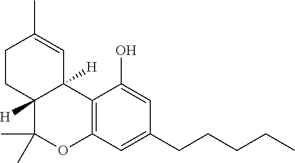 [SMILES: CCCCCc1cc(O)c2c(c1)OC(C)(C)[C@@H]1CCC(C)=C[C@@H]21] |

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the Cannabis plant. The phytocannabinoids can be isolated from plants to produce a highly purified extract or can be reproduced synthetically.

“Highly purified cannabinoid extracts” are defined as cannabinoids that have been extracted from the Cannabis plant and purified to the extent that other cannabinoids and non-cannabinoid components that are co-extracted with the cannabinoids have been substantially removed, such that the highly purified cannabinoid is greater than or equal to 98% (w/w) pure.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“Childhood epilepsy” refers to the many different syndromes and genetic mutations that can occur to cause epilepsy in childhood. Examples of some of these are as follows: Dravet Syndrome; Myoclonic-Absence Epilepsy; Lennox-Gastaut syndrome; Generalized Epilepsy of unknown origin; CDKL5 mutation; Aicardi syndrome; bilateral polymicrogyria; Dup15q; SNAP25; and febrile infection related epilepsy syndrome (FIRES); benign rolandic epilepsy; juvenile myoclonic epilepsy; infantile spasm (West syndrome); and Landau-Kleffner syndrome. The list above is non-exhaustive as many different childhood epilepsies exist.

“Focal Seizures” are defined as seizures which originate within networks limited to only one hemisphere. What happens during the seizure depends on where in the brain the seizure happens and what that part of the brain normally does.

“Focal seizure where awareness/consciousness are impaired” has replaced the term “complex partial seizure”. These seizures usually start in a small area of the temporal lobe or frontal lobe of the brain and involve other areas of the brain within the same hemisphere that affect alertness and awareness. Most subjects experience automatisms during a focal seizure with impaired consciousness.

“Mixed seizures” are defined as the existence of both generalised and focal seizures in the same patient.

The terms “50% responder” and “50% reduction in seizure” are both terms used in clinical studies. In the present application the terms define the percentage of subjects that experienced a greater than or equal to 50% reduction in the number of seizures during treatment with CBD in comparison to the number experienced during the baseline period before the CBD was administered.

### DETAILED DESCRIPTION

### Preparation of Highly Purified CBD Extract

The following describes the production of the highly-purified (>98% w/w) cannabidiol extract which has a known and constant composition which was used for the expanded access trials described in the Examples below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 98% CBD.

The Cannabis sativa L. plants are grown, harvested, and processed to produce a botanical extract (intermediate) and then purified by crystallization to yield the CBD (drug substance).

The plant starting material is referred to as Botanical Raw Material (BRM); the botanical extract is the intermediate; and the active pharmaceutical ingredient (API) is CBD, the drug substance.

Both the botanical starting material and the botanical extract are controlled by specifications. The drug substance specification is described in Table 1 below.

TABLE 5
CBD Specification

| Test | Test Method | Limits |
|---|---|---|
| Appearance | Visual | Off-white/pale yellow crystals |
| Identification A | HPLC-UV | Retention time of major peak |
| | | corresponds to certified CBD |
| | | Reference Standard |
| Identification B | GC-FID/MS | Retention time and mass |
| | | spectrum of major peak |
| | | corresponds to certified |
| | | CBD Reference Standard |
| Identification C | FT-IR | Conforms to reference spectrum |
| | | for certified CBD Reference |
| | | Standard |
| Identification D | Melting | 65-67° C. |
| | Point | |
| Identification E | Specific | Conforms with certified CBD |
| | Optical | Reference Standard; −110° |
| | Rotation | to −140° (in 95% ethanol) |
| Total Purity | Calculation | ≥98.0% |
| Chromatographic Purity | HPLC-UV | ≥98.0% |
| 1 | | |
| Chromatographic Purity | GC-FID/MS | ≥98.0% |
| 2 | | |
| Other Cannabinoids: | HPLC-UV | |
| CBDA | | NMT 0.15% w/w |
| CBDV | | NMT 1.0% w/w |
| Δ⁹ THC | | NMT 0.15% w/w |
| CBD-C4 | | NMT 0.5% w/w |
| Residual Solvents: | GC | |
| Alkane | | NMT 0.5% w/w |
| Ethanol | | NMT 0.5% w/w |
| Residual Water | Karl Fischer | NMT 1.0% w/w |

NMT—Not more than

The purity of the CBD drug substance achieved is greater than 98%. The other cannabinoids which may occur in the extract are: CBDA, CBDV, CBD-C4 and THC.

Distinct chemotypes of Cannabis sativa L. plant have been produced to maximize the output of the specific chemical constituents, the cannabinoids. One type of plant produces predominantly CBD. Only the (−)-trans isomer occurs naturally. Furthermore during purification the stereochemistry of CBD is not affected.

### Production of the Intermediate

An overview of the steps to produce a botanical extract, the intermediate, are as follows:
1. Growing
2. Decarboxylation
3. Extraction No.1—using liquid CO₂
4. Extraction No.2—‘winterization’ using ethanol
5. Filtration
6. Evaporation

High CBD chemovars were grown, harvested and dried and stored in a dry room until required. The botanical raw material (BRM) was finely chopped using an Apex mill fitted with a 1 mm screen. The milled BRM was stored in a freezer for up to 3 months prior to extraction.

Decarboxylation of CBDA to CBD was carried out using a large Heraeus tray oven. The decarboxylation batch size in the Heraeus is approximately 15 Kg. Trays were placed in the oven and heated to 105° C.; the BRM took 96.25 minutes to reach 105° C. Held at 105° C. for 15 Minutes. Oven then set to 150° C. . . . ; the BRM took 75.7 minutes to reach 150° C.; BRM held at 150° C. for 130 Minutes. Total time in the oven was 380 Minutes, including 45 minutes cooling and 15 Minutes venting.

Extraction No 1 was performed using liquid CO₂ at 60 bar/10° C. to produce botanical drug substance (BDS).

The crude CBD BDS was winterised in Extraction No 2 under standard conditions (2 volumes of ethanol at minus 20° C. for around 50 hours). The precipitated waxes were removed by filtration and the solvent evaporated using the rotary evaporator (water bath up to 60° C.) to yield the BDS, which was then used for crystallisation to produce the test material.

### Production of the Drug Substance

The manufacturing steps to produce the drug substance from the intermediate botanical extract are as follows:
1. Crystallization using C5-C12 straight chain or branched alkane
2. Filtration
3. Optional recrystallization from C5-C12 straight chain or branched alkane
4. Vacuum drying

Intermediate botanical extract (12 kg) produced using the methodology above was dispersed in C5-C12 straight chain or branched alkane (9000 ml, 0.75 vols) in a 30 litre stainless steel vessel.

The mixture was manually agitated to break up any lumps and the sealed container then placed in a freezer for approximately 48 hours.

The crystals were isolated by vacuum filtration, washed with aliquots of cold C5-C12 straight chain or branched alkane (total 12000 ml), and dried under a vacuum of <10 mb at a temperature of 60° C. until dry before submitting the drug substance for analysis.

The dried product was stored in a freezer at minus 20° C. in a pharmaceutical grade stainless steel container, with FDA food grade approved silicone seal and clamps.

### Production of the Drug Product

The drug product is presented as an oral solution. The oral solution presentation contains 25 mg/ml or 100 mg/ml CBD, with the excipients sesame oil, ethanol, sucralose and flavouring. Two product strengths are available to allow dose titration across a wide dose range.

The 25 mg/ml solution is appropriate at lower doses and the 100 mg/ml solution at higher doses.

The drug product formulation is as described in Table 6 below:

TABLE 6
Drug Product specification

| | | | Reference |
|---|---|---|---|
| | Qualitative | | to Quality |
| Component | Composition | Function | Standard |
| Cannabidiol (CBD) | 25 mg/ml or 100 mg/ml | Active | In-house |
| Anhydrous ethanol | 79.0 mg/ml* | Excipient | Ph.Eur. |
| Sucralose | 0.5 mg/ml | Sweetener | In-house |
| Strawberry | 0.2 mg/ml | Flavouring | In-house |
| flavouring | | | |
| Sesame oil | q.s to 1.0 ml | Excipient | Ph.Eur. |

The drug substance, CBD is insoluble in water. Sesame oil was selected as an excipient to solubilize the drug substance.

A sweetener and fruit flavouring are required to improve palatability of the sesame oil solution.

Ethanol was required to solubilize the sweetener and the flavouring.

The composition can be substantially equivalent, by which is meant the functional ingredients can vary from the qualitative composition specified in Table 6 by an amount of up to 10%.

Example 1 below describes the use of a highly purified Cannabis extract comprising cannabidiol (CBD). Cannabidiol is the most abundant non-psychoactive cannabinoid in the selected chemovar. Previous studies in animals have demonstrated that CBD has anticonvulsant efficacy in multiple species and models.

Example 1 describes data produced in an expanded access treatment program in children with TRE.

### Example 1: Efficacy of Cannabidiol Reducing Focal Seizures in Children and Young Adults with Intractable Epilepsy

### Materials and Methods

Of 137 children and young adults with severe, childhood onset treatment-resistant epilepsy (TRE), fifty-one suffered from epilepsy that was characterised by focal seizures. These subjects were tested with a highly purified extract of cannabidiol (CBD) obtained from a Cannabis plant. All subjects presented with focal type seizures, often in addition to generalised seizures. The participants in the study were part of an expanded access compassionate use program for CBD.

The epileptic syndromes that these patients suffered from were as follows: Lennox-Gastaut Syndrome; Tuberous Sclerosis Complex; Dravet Syndrome; CDKL5; Neuronal ceroid lipofuscinoses (NCL); febrile infection related epilepsy syndrome (FIRES); Aicardi syndrome and brain abnormalities.

Other seizure types experienced by these patients included: tonic, clonic, tonic-clonic, myoclonic, atonic, absence, myoclonic-absence, focal seizures without impairment, focal seizures with impairment and focal seizures evolving to bilateral convulsive seizures.

All patients entered a baseline period of 4 weeks when parents/caregivers kept prospective seizure diaries, noting all countable seizure types.

The patients then received a highly purified CBD extract (greater than 98% CBD w/w) in sesame oil, of known and constant composition, at a dose of 5 mg/kg/day in addition to their baseline anti-epileptic drug (AED) regimen.

The daily dose was gradually increased by 2 to 5 mg/kg increments until intolerance occurred or a maximum dose of 25 mg/kg/day was achieved.

Patients were seen at regular intervals of 2-4 weeks. Laboratory testing for hematologic, liver, kidney function and concomitant AED levels was performed at baseline, and after every 4 weeks of CBD therapy.

The patients on the study were all taking at least one concomitant AED. These included: carbamezapine, clobazam, clonazepam, clonidine, clorazepate, desmethylclobazam, diazepam, ethosuximide, felbamate, ketogenic diet, lacosamide, lamotrigine, levetiracetam, lorazepam, midazolam, N-desmethylclobazam, nordiazepam, oxycarbamezapine, perampanel, phenobarbital, phenytoin, pregabalin, rufinamide, stiripentol, topiramate, trazodone, vagus nerve stimulation, valproic acid, vigabatrin, and zonisamide.

### Results

The 51 children and young adult patients all of whom suffered from focal seizures received treatment with CBD who received treatment for at least 12 weeks.

A summary of the 50% responders, based on 12 weeks of treatment are summarized in Table 7 below.

TABLE 7
Summary of 50% responders after 12 weeks
of treatment for focal seizures

| | Focal seizures | Total seizures |
|---|---|---|
| | (n = 51) | (n = 137) |
| >50% reduction in | 63% (n = 32) | 46% (n = 63) |
| seizures | | |
| <50% reduction in | 37% (n = 19) | 54% (n = 74) |
| seizures | | |

Table 7 shows that after 3 months of therapy, a remarkable 63% of patients had an equal to or greater than >50% reduction in focal seizures, these data infer that the CBD is very effective at reducing this type of seizure.

### Conclusions

These data indicate that CBD significantly reduces the number of focal seizures in a high proportion of patients that do not respond well to existing AED.

It was surprising that in this group of patients which are treatment-resistant such a high number were able to gain an effect. The fact that nearly two thirds of the patients (63%) benefitted from at least a fifty percent reduction in the number of focal seizures that they suffered from was remarkable.

### Example 2: Efficacy of Cannabidiol Reducing Focal Seizures with Impairment in Children and Young Adults with Intractable Epilepsy

### Materials and Methods

Of 137 children and young adults with severe, childhood onset treatment-resistant epilepsy (TRE), thirty-seven suffered from epilepsy that was characterised by focal seizures with impairment. These subjects were tested with a highly purified extract of cannabidiol (CBD) obtained from a Cannabis plant. All subjects presented with focal seizures with impairment, often in addition to other generalised and/or focal seizures. The participants in the study were part of an expanded access compassionate use program for CBD.

The epileptic syndromes that these patients suffered from were as follows: Lennox-Gastaut Syndrome; Tuberous Sclerosis Complex; Dravet Syndrome; CDKL5; febrile infection related epilepsy syndrome (FIRES); Aicardi syndrome and brain abnormalities.

All patients entered a baseline period of 4 weeks when parents/caregivers kept prospective seizure diaries, noting all countable seizure types.

The patients then received a highly purified CBD extract (greater than 98% CBD w/w) in sesame oil, of known and constant composition, at a dose of 5 mg/kg/day in addition to their baseline anti-epileptic drug (AED) regimen.

The daily dose was gradually increased by 2 to 5 mg/kg increments until intolerance occurred or a maximum dose of 25 mg/kg/day was achieved.

Patients were seen at regular intervals of 2-4 weeks. Laboratory testing for hematologic, liver, kidney function and concomitant AED levels was performed at baseline, and after every 4 weeks of CBD therapy.

The patients on the study were all taking at least one concomitant AED. These included: carbamezapine, clobazam, clonazepam, clorazepate, desmethylclobazam, diazepam, ethosuximide, felbamate, ketogenic diet, lacosamide, lamotrigine, levetiracetam, lorazepam, midazolam, N-desmethylclobazam, nordiazepam, oxycarbamezapine, perampanel, phenobarbital, phenytoin, pregabalin, rufinamide, topiramate, vagus nerve stimulation, valproic acid, vigabatrin, and zonisamide.

### Results

The 37 children and young adult patients all of whom suffered from focal seizures with impairment received treatment with CBD who received treatment for at least 12 weeks.

A summary of the 50% responders, based on 12 weeks of treatment are summarized in Table 8 below.

TABLE 8
Summary of 50% responders after 12 weeks of
treatment for focal seizures with impairment

| | Focal Seizures with | Total seizures |
|---|---|---|
| | Impairment (n = 37) | (n = 137) |
| >50% reduction in | 65% (n = 24) | 46% (n = 63) |
| seizures | | |
| <50% reduction in | 35% (n = 13) | 54% (n = 74) |
| seizures | | |

Table 8 shows that after 3 months of therapy, a remarkable 65% of patients had an equal to or greater than >50% reduction in focal seizures with impairment, these data infer that the CBD is very effective at reducing this type of seizure.

Furthermore when these data are compared to the other sub-types of focal seizure, namely focal seizure without impairment and focal seizures leading to secondary generalisation it can clearly be seen that CBD was able to selectively reduce the occurrence of focal seizures with impairment. Table 9 below details these findings.

TABLE 9
Summary of 50% responders after 12 weeks
of treatment for all focal seizure types

| | | | Focal | |
|---|---|---|---|---|
| | Focal | Focal | Seizures | |
| | Seizures | Seizures | Leading to | Total |
| | with | without | Secondary | Focal |
| | Impairment | Impairment | Generalised | seizures |
| | (n = 37) | (n = 6) | (n = 15) | (n = 51) |
| >50% reduction | 65% | 50% | 47% | 63% |
| in seizures | (n = 24) | (n = 3) | (n = 7) | (n = 32) |
| <50% reduction | 35% | 50% | 53% | 37% |
| in seizures | (n = 13) | (n = 3) | (n = 8) | (n = 19) |

### Conclusions

These data indicate that CBD significantly reduces the number of focal seizures with impairment in a selective manner.

It was surprising that in this group of patients which are treatment-resistant such a high number were able to gain an effect. The fact that over two thirds of the patients (65%) benefitted from at least a fifty percent reduction in the number of focal seizures with impairment that they suffered from was remarkable.

### REFERENCES

Ames F R and Cridland S (1986). “Anticonvulsant effects of cannabidiol.” S Afr Med J 69:14.
Consroe P, Martin P, Eisenstein D. (1977). “Anticonvulsant drug antagonism of delta-9-tetrahydrocannabinol induced seizures in rabbits.” Res Commun Chem Pathol Pharmacol. 16:1-13
Consroe P, Benedicto M A, Leite J R, Carlini E A, Mechoulam R. (1982). “Effects of cannabidiol on behavioural seizures caused by convulsant drugs or current in mice.” Eur J Pharmaco. 83:293-8
Cunha J M, Carlini E A, Pereira A E, Ramos O L, Pimental C, Gagliardi R et al. (1980). “Chronic administration of cannabidiol to healthy volunteers and epileptic patient.” Pharmacology. 21:175-85
Dravet C. The core Dravet syndrome phenotype. Epilepsia. 2011 April: 52 Suppl 2:3-9.
Eadie, M J (December 2012). “Shortcomings in the current treatment of epilepsy.” Expert Review of Neurotherapeutics 12 (12): 1419-27.
Kwan P, Arzimanoglou A, Berg A T, Brodie M J, Hauser W A, Mathern G, Moshé S L, Perucca E, Wiebe S, French J. (2009) “Definition of drug resistant epilepsy: Consensus proposal by the ad hoc Task Force of the ILAE Commission on Therapeutic Strategies.” Epilepsia.
Mechoulam R and Carlini E A (1978). “Toward drugs derived from Cannabis.” Die naturwissenschaften 65:174-9.
Porter B E, Jacobson C (December 2013). “Report of a parent survey of cannabidiol-enriched Cannabis use in paediatric treatment resistant epilepsy” Epilepsy Behaviour. 29(3) 574-7
Thurman, D J; Beghi, E; Begley, C E; Berg, A T; Buchhalter, J R; Ding, D; Hesdorffer, D C; Hauser, W A; Kazis, L; Kobau, R; Kroner, B; Labiner, D; Liow, K; Logroscino, G; Medina, M T; Newton, C R; Parko, K; Paschal, A; Preux, P M; Sander, J W; Selassie, A; Theodore, W; Tomson, T; Wiebe, S; ILAE Commission on, Epidemiology (September 2011). “Standards for epidemiologic studies and surveillance of epilepsy.” Epilepsia. 52 Suppl 7:2-26

1-20. (canceled)

21. A method of treating focal seizures in a subject having treatment resistant epilepsy, comprising administering to the subject a drug product comprising a cannabidiol (CBD) drug substance, wherein the CBD drug substance has a purity of at least 98% (w/w), wherein the CBD is administered at a dose ranging from about 5 mg/kg/day to about 25 mg/kg/day.

22. The method of claim 21, wherein the focal seizures are focal seizures with impairment.

23. The method of claim 21, wherein the epilepsy is Lennox-Gastaut Syndrome, Tuberous Sclerosis Complex, Dravet Syndrome; CDKL5, Neuronal ceroid lipofuscinoses (NCL), febrile infection related epilepsy syndrome (FIRES) or Aicardi syndrome.

24. The method of claim 21, wherein the CBD is administered in combination with one or more concomitant anti-epileptic drugs (AED).

25. The method of claim 24, wherein the one or more AED is selected from the group consisting of: carbamezapine, clobazam, clonazepam, clonidine, clorazepate, desmethylclobazam, diazepam, ethosuximide, felbamate, ketogenic diet, lacosamide, lamotrigine, levetiracetam, lorazepam, midazolam, N-desmethylclobazam, nordiazepam, oxycarbamezapine, perampanel, phenobarbital, phenytoin, pregabalin, rufinamide, stiripentol, topiramate, trazodone, vagus nerve stimulation, valproic acid, vigabatrin, and zonisamide.

26. The method of claim 21, wherein the CBD is a highly purified extract of Cannabis.

27. The method of claim 26, wherein the highly purified extract further comprises less than 0.15% tetrahydrocannabinol (THC).

28. The method of claim 26, wherein the highly purified extract further comprises up to 1% (w/w) cannabidivarin (CBDV).

29. The method of claim 21, wherein the CBD is present as a synthetic compound.

30. The method of claim 21, wherein the dose of the CBD is increased to about 10 mg/kg/day.

31. The method of claim 21, wherein the dose of the CBD is increased to about 15 mg/kg/day.

32. The method of claim 21, wherein the dose of the CBD is increased to about 20 mg/kg/day.

33. The method of claim 21, wherein the dose of the CBD is increased to about 25 mg/kg/day.

34. The method of claim 21, comprising administering the drug product comprising the CBD drug substance for at least 12 weeks.

35. The method of claim 34, wherein the focal seizures are reduced by at least 50% compared to baseline.

36. The method of claim 35, wherein the focal seizures are focal seizures with impairment.

37. The method of claim 35, wherein the focal seizures are focal seizures without impairment.

38. The method of claim 35, wherein the focal seizures are focal seizures leading to secondary generalized seizures.

39. The method of claim 35, wherein the dose of the CBD is increased to about 10 mg/kg/day.

40. The method of claim 35, wherein the dose of the CBD is increased to about 15 mg/kg/day.

41. The method of claim 35, wherein the dose of the CBD is increased to about 20 mg/kg/day.

42. The method of claim 35, wherein the dose of the CBD is increased to about 25 mg/kg/day.

43. The method of claim 36, wherein the dose of the CBD is increased to about 10 mg/kg/day.

44. The method of claim 36, wherein the dose of the CBD is increased to about 15 mg/kg/day.

45. The method of claim 36, wherein the dose of the CBD is increased to about 20 mg/kg/day.

46. The method of claim 36, wherein the dose of the CBD is increased to about 25 mg/kg/day.
